(19) United States
(12) Patent Application Publication
(10) Pub. No.: US 2012/0249955 A1
(43) Pub. Date: Oct. 4, 2012

(54) Retro-illumination and eye front surface feature registration for corneal topography and ocular wavefront system

(76) Inventors: Edwin J. Sarver, Carbondale, IL (US); Donald R. Sanders, Elmhurst, IL (US)

(21) Appl. No.: 13/428,688

(22) Filed: Mar. 23, 2012

Related U.S. Application Data
(60) Provisional application No. 61/467,229, filed on Mar. 24, 2011.

(51) Int. Cl. A61B 3/14

(52) U.S. Cl. 351/206; 351/246

(57) ABSTRACT

A method of obtaining a retro-illumination image using the beacon from an ocular wavefront path and the camera for the corneal topography path of the combined system. A digital image of the retro-illuminated view of the IOL, iris pattern and sclera is obtained. An interactive display of the retro-illuminated image is presented to the user to allow them to identify the orientation marks on the IOL. These marks identify the orientation of the IOL and an overlay line can be used to display this orientation. In addition, a 360 degree overlay can be used to enhance the display of this orientation line.

### RELATED APPLICATIONS

This application claims the benefit of priority under 35 U.S.C. 119(e) of U.S. Provisional Patent Application No. 61/467,229, filed Mar. 24, 2011, entitled, “RETROILLUMINATION AND EYE FRONT SURFACE FEATURE REGISTRATION FOR CORNEAL TOPOGRAPHY AND OCULAR WAVEFRONT SYSTEM”, the entirety of which is incorporated herein by reference.

### FIELD OF THE INVENTION

The present invention relates to systems and methods for human vision correction, and in particular, to a method of retro-illumination and eye front surface feature registration.

### BACKGROUND OF THE INVENTION

Corneal topography systems provide a detailed surface description of the front of the eye's corneal surface in a mathematical form. Ocular wavefront systems provide a detailed description of the optical state for the entire eye. Together these optometric and ophthalmic systems provide information of the eye's optical errors and how to correct them using a wide range of methods including spectacles, contact lenses, corneal refractive surgery, and phakic and aphakic intraocular lenses (IOLs).

It is well known that, by subtracting the corneal wavefront aberrations (computed from corneal topography) from the ocular wavefront aberrations, a representation of the internal aberrations of the eye can be obtained. In particular, if the aphakic eye (eye without a natural crystalline lens) has a toric IOL, it is of interest to ensure that the principal axes of the IOL are positioned to optimally correct the eye's astigmatism arising primarily at the cornea. By determining the internal aberrations using the subtraction described above, an estimate of the IOL's cylinder axis can be obtained. If the toric IOL cylinder is not oriented in the correct axis, the IOL may need to be rotated to provide high quality correction for the eye. If the internal aberrations are accurate, they can be used to help determine the angular amount and direction required to correct a toric IOL cylinder orientation error.

In some cases the corneal aberrations or the ocular aberrations may not be accurately determined, for example, due to image processing errors. In these cases the internal aberrations will not be accurately determined and thus, the toric IOL's cylinder axis will also not be accurately determined. To help provide accurate toric IOL orientation information, a view of the toric IOL within the eye is useful. Since toric IOLs have marks indicating the lens orientation within the eye, locating these marks identifies the IOLs orientation. A suitable view of the IOLs can be obtained by placing a point of light on the retina, and viewing the IOL from the front as the light exits the eye and fills the entrance pupil. The camera should be focused on the entrance pupil for this viewing. This viewing geometry is referred to as retro-illumination since the IOL is illuminated from the “back”. In the present invention, this view is obtained using the beacon from the ocular wavefront path and the camera for the corneal topography path of the combined system. A digital image of the retro-illuminated view of the IOL is captured and at the same time, an image of the front of the eye (primarily iris pattern and sclera) is obtained. A sample image of a retro-illuminated toric IOL within the eye is shown in FIG. 1.

An interactive display of the retro-illuminated image is presented to the user to allow them to identify the orientation marks on the IOL. These marks identify the orientation of the IOL and an overlay line can be used to display this orientation. In addition, a 360 degree overlay can be used to enhance the display of this orientation line. A sample retro-illuminated toric IOL with the marks, orientation line, and 360 degree graphic overlays is shown in FIG. 2.

A question that must be addressed in providing the information such as toric IOL cylinder axis, concerns the cyclorotation of the eye or head tilt between exams. It is known that the eye will rotate about its optical axis (called a cyclorotation) between exams. This rotation is illustrated in FIG. 3. In a study by Wolffsohn and Buckhurst, (Objective analysis of toric intraocular lens rotation and centration, J Cataract Refract Surg, 2010, 36(5):778-82), the mean cyclorotation between exams was found to be about 2.2 degrees with a standard deviation of about 1.8 degrees. Thus, we would like to remove this type of rotation from our measurement of (for example) toric IOL cylinder axis.

### SUMMARY OF THE INVENTION

In our strategy we find features on the eye's front surface (naturally occurring in the iris pattern or sclera or artificially placed on the sclera with marks) that occur in two images, for example, pre- and post-surgery. By comparing the point correspondences between the two sets of features in the images, we can determine the rotation angle between them. This cyclorotation angle can then be used to correct the reporting of eye measurement at certain axes.

In FIG. 4 we show the front view of the eye with the retro-illuminated light source turned off. In this eye the iris patterns show up well. In other eyes, some veins can be seen in the sclera. It is also possible to place artificial marks on the sclera to identify a specific meridian. By considering the automatically calculated limbus contour and outer most ring, we can define a region where “good” registration features can be automatically selected from the iris image. The definition of “good” registration features are those that have nearly perpendicular image gradients. An example of this would be a “corner like” feature in an image. In addition to these automatically detected features, the user can interactively select points of interest from the sclera such as blood vessel forks or artificially placed marks. The set of automatic and interactively selected points can then be stored with the exam image. As other exams are captured, these features from the “reference” image can be automatically compared to the exam image to be registered. This is illustrated in FIG. 5. The point mapping from the reference to the exam image to be registered, can also be interactively edited to ensure that the rotation angle between the two sets of images is accurate.

Another application of this retro-illumination would be to capture a live image through an operative microscope. The same software would be employed to allow the user to identify the tonic IOL marks and then generate the orientation graphic display. This would be of great help to the surgeon at the time of surgery. The reference image/registered image function using a preoperative exam could also be used to report the toric IOL axis relative to the preoperative reference image and features.

These and other objectives and advantages of this invention will become apparent from the following description taken in conjunction with any accompanying drawings wherein are set forth, by way of illustration and example, contain embodiments of this invention. Any drawings contained herein constitute a part of this specification and include exemplary embodiments of the present invention and illustrate various objects and features thereof.

### BRIEF DESCRIPTION OF THE DRAWINGS

FIG. 1. Photo of an retro-illuminated toric IOL in the eye illuminated with NIR light;

FIG. 2. Photo of FIG. 1 with the axis identified by interactively placed spots and a 360 degree graphic;

FIG. 3. Pictorial of a cyclorotation of the eye about the eye's longitudinal axis;

FIG. 4. Photo of an Iris image captured at the same time as the retro-illuminated toric IOL image;

FIG. 5. Photos identifying features in the iris pattern or on the sclera;

FIG. 6. Basic layout for retro-illumination and front eye image features acquisition;

FIG. 7. General tab for retro-illumination editor;

FIG. 8. Display tab for retro-illumination editor;

FIG. 9. Image tab for retro-illumination editor; and

FIG. 10. Spots tab for retro-illumination editor.

### DETAILED DESCRIPTION OF THE PREFERRED EMBODIMENT

The basic optical layout for the retro-illumination and the front eye image features acquisition is shown in FIG. 6. Here, a near IR beam of light originates at SLD 1 and is directed though beam splitter 2 toward the eye 3. This beam forms a diffuse reflection at the retina and the light is directed out of the eye filling the entrance pupil and back illuminating the toric IOL within the eye. Light from the back lit toric IOL is directed by beam splitter 2 toward the lens 4 and forms an image on the camera sensor 5. This is the retro-illuminated image of the toric IOL. Immediately after the retro-illuminated image is captured, the SLD 1 is turned off, and the near IR LEDs 6 forming the light for the corneal topography rings are turned on. This light reflects off the front of the eye including the iris and sclera and is directed by beam splitter 2 to lens 4 and also forms an image on camera sensor 5. This second image is thus captured within a camera frame duration (typically 33 mS) from the time the retro-illuminated image was captured.

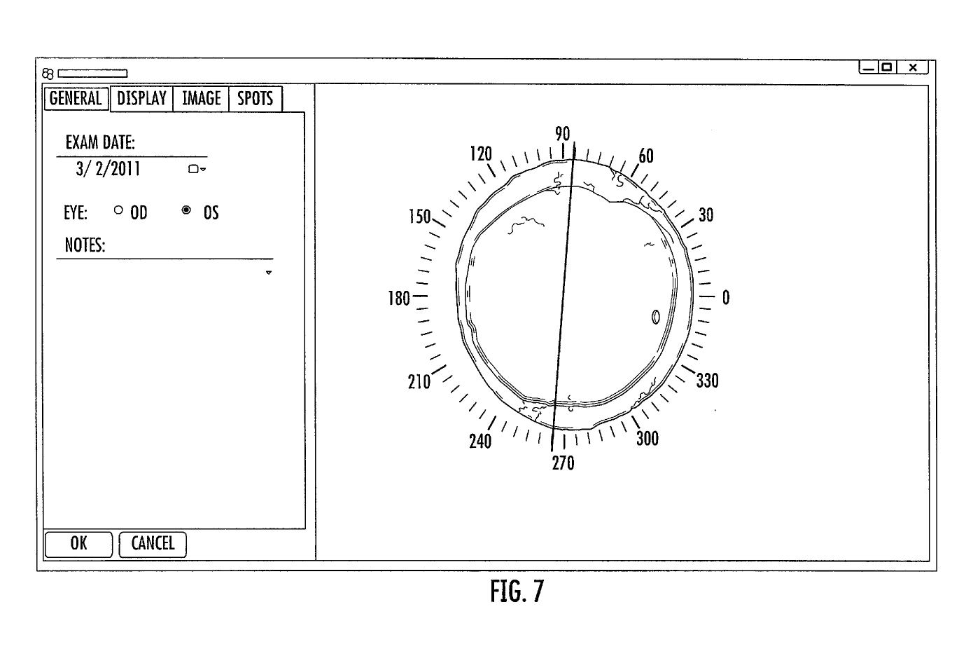

Once the retro-illuminated toric IOL image is acquired, it can be edited to locate the cylinder axis of the IOL. The four tabs of the retro-illumination editor are shown in FIGS. 7-10. On the General tab, the date of the exam, which eye the exam is for, and a note can be viewed or edited. On the Display tab, the user can show or hide the meridian graphic overlay using the Show meridian check box. The text showing the axis in degrees can be placed in the middle of the graphic or at the edge of the meridian using the Axis text location group box. The resolution of the reported axis angle can be adjusted from 0.1, 0.25, 0.5, or 1 degree by using the Axis rounding pick list box. The Image tab allows the user to adjust the image brightness, contrast, sharpness, or smoothness so that details of the toric IOL can be better seen. At any point, the image can be returned to its original condition using the Restore button. An enhanced image will be saved with the exam for viewing later in the general displays (separate from the editor). The Spots tab allows the user to add, delete, and show the spots that will define the cylinder axis of the toric IOL. The Use spot constraints check box forces the meridian graphic to pass through the spots placed by the user. The editor also allows the display to be zoomed and panned to get a better view of specific regions of interest in the image. The edited data is saved with the exam for later display.

The second image acquired is used to align the retro-illumination image data to another exam using features common to both images. There are two methods used to define these features. The first is automatically by searching for “corner-like” features in the region between the last ring and the limbus contour. This is a common image processing task known to those skilled in the art. The second method is an interactive method when the user places spots on the image to identify neighborhoods that contain the features. This interactive process is exactly like that described above for the retro-illumination editor so is not described again here. These features are saved with the exam and are used to determine how the exam is registered (via cyclorotation angle only—not translation) to another exam.

For the intraoperative application, the retro-illumination image is acquired from a digital camera or videorecorder attached to the surgical microscope. The digital image is transferred to the software program where the retro-illumination editor is used to measure the orientation axis of the toric IOL.

Obvious extensions of the method include:

1. The analysis of the retro-illumination image can be extended to include phakic toric IOLs, custom IOLs, multifocal IOL, or other optical or mechanical features in the eye.

2. The retro-illumination image can be used to measure and document features such as cataract or other ocular changes at the IOL/crystalline plane.

3. The orientation features (three dots, lines, diamonds, etc) used by IOLs to indicate cylinder axis could be automatically found using feature matching techniques known to those skilled in the art.

4. The orientation of the IOLs and desired directions and astigmatic calculations could be performed and displayed on the corneal topography, ocular wavefront, or other display for the user and/or patient to view.

5. The retro-illumination function could be part of an IOL planner/evaluation system based upon the image, the corneal topography, and the ocular wavefront. This could include additional external data such as that provided by axial length measurement systems.

6. The illumination for the iris could be either NIR or visible depending upon the application. If the retro-illumination/iris image pair are to be compared to an externally acquired iris image that used visible illumination for the iris image, then the system would perform better in some circumstances if the iris illumination were also visible. For example, certain details of the iris and blood vessels in the sclera are better imaged in visible light.

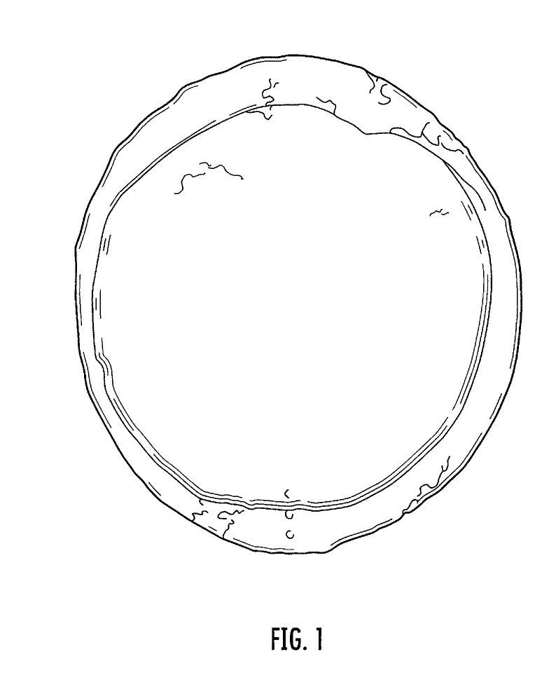

FIG. 1. A retro-illuminated toric IOL in the eye illuminated with NIR light. The orientation marks for this particular IOL are three dots at each end of the cylinder axis.

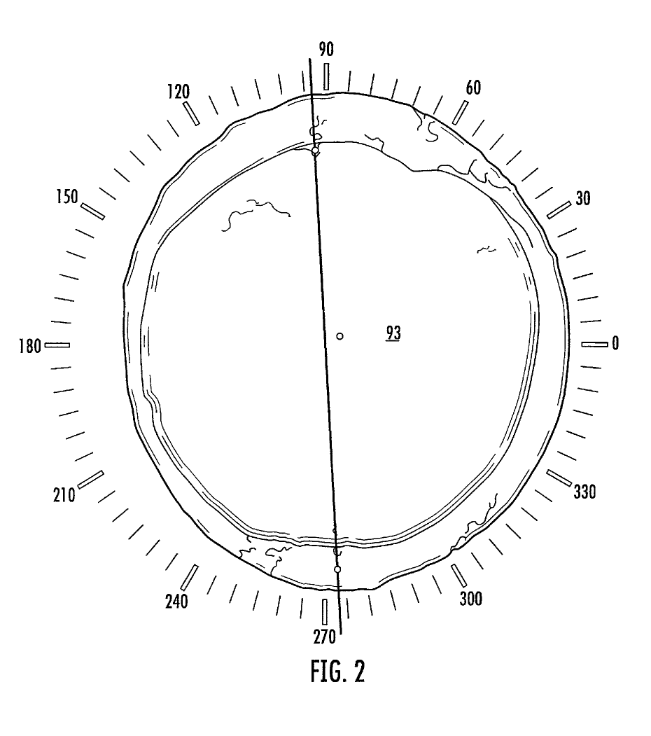

FIG. 2. This is the same retro-illuminated image of the toric IOL with the axis identified by two interactively placed spots (white circles with black border) at the top and bottom of the image. The orientation of the meridian at 93 degrees and is indicated on the display. The 360 degree graphic also helps the user visualize the orientation of the toric IOL cylinder axis.

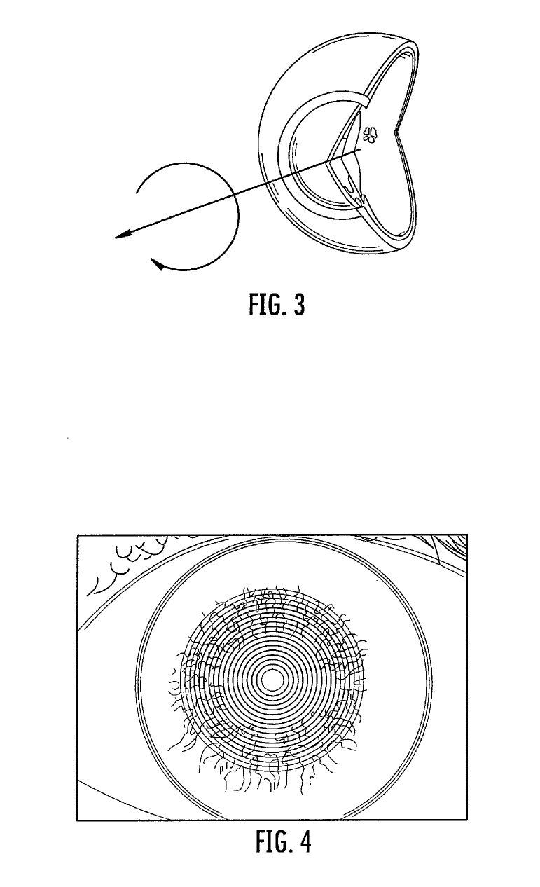

FIG. 3. Cyclorotation of the eye about the eye's longitudinal axis.

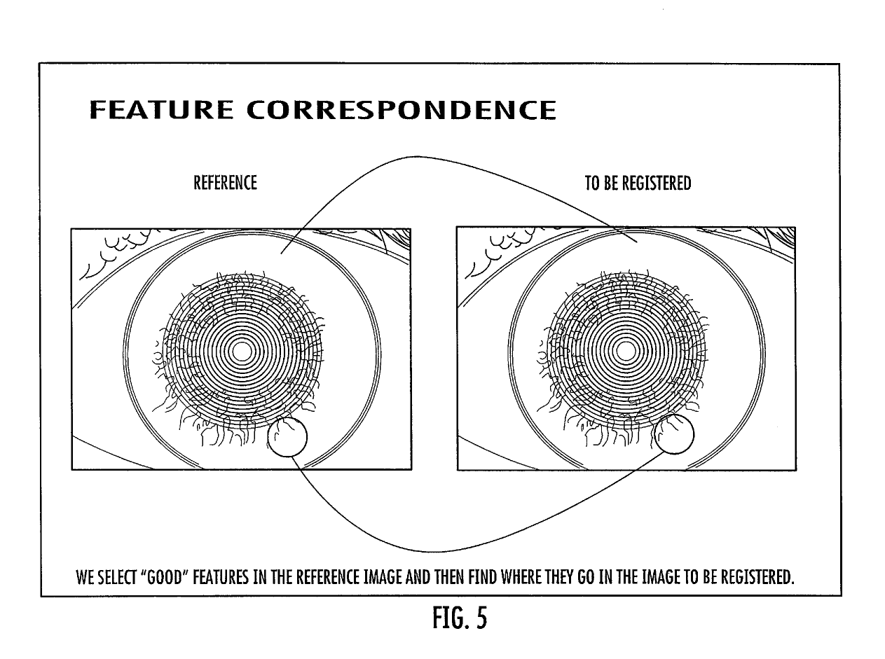

FIG. 4. Iris image captured at the same time as the retro-illuminated toric IOL image. This image is captured with the wavefront beacon turned off (no retro-illumination) and the corneal topography rings turned on. This works because the eye's entrance pupil for the retro-illumination image is located at nearly the same focal plane as the iris plane so that both are in focus at the same time.

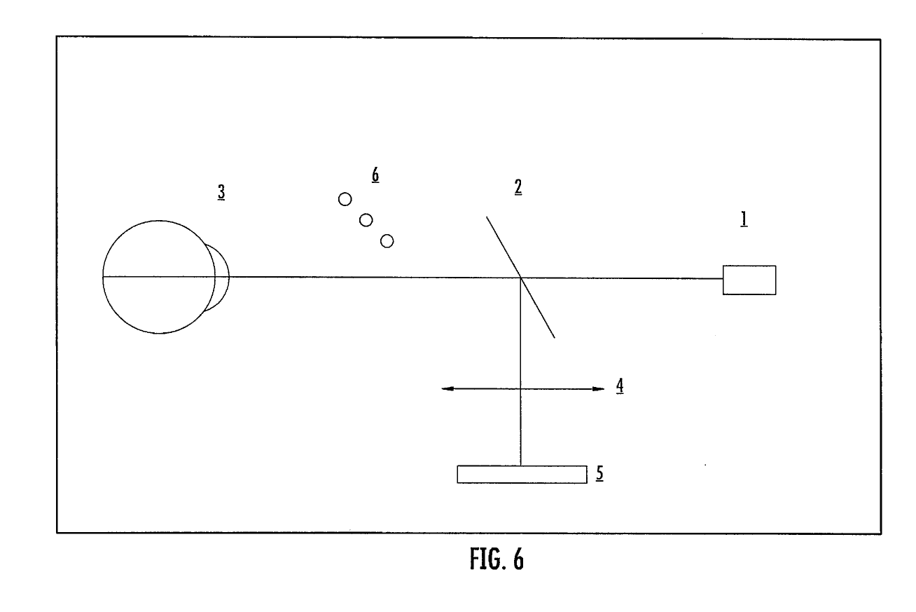

FIG. 5. We can identify features in the iris pattern or on the sclera, and determine where they go in another image of the same eye. In this way we can determine the rotation of the eye between image captures. We can thus align data between two eye exams taken some time apart or pre- and post-surgery to ensure we attribute axis measurements (such as IOL cylinder axis) to the lens and not to the eye's cyclorotation.

FIG. 6. Basic layout for retro-illumination and front eye image features acquisition.

FIG. 7. General tab for retro-illumination editor.

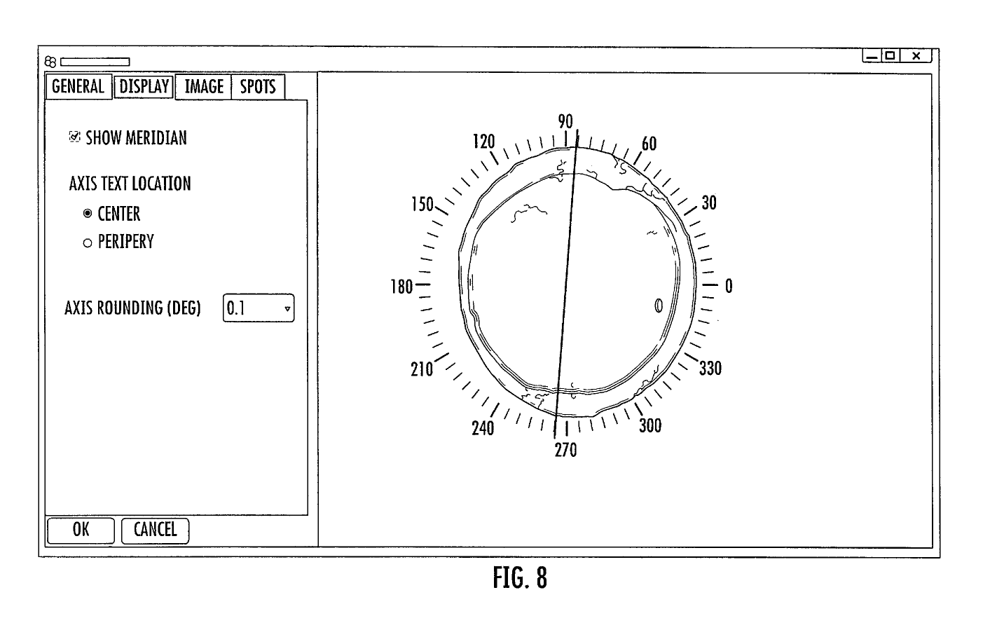

FIG. 8. Display tab for retro-illumination editor.

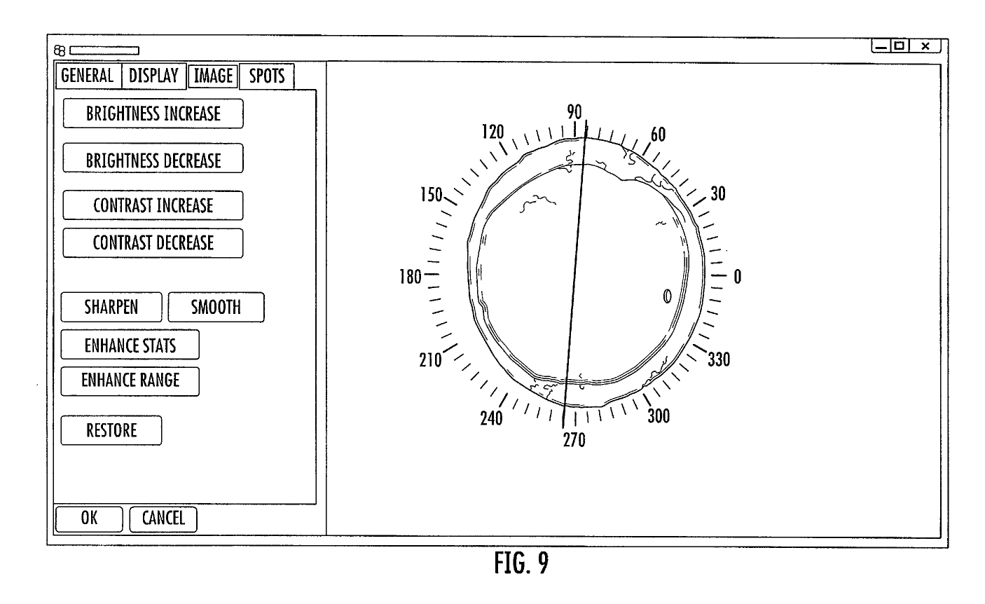

FIG. 9. Image tab for retro-illumination editor.

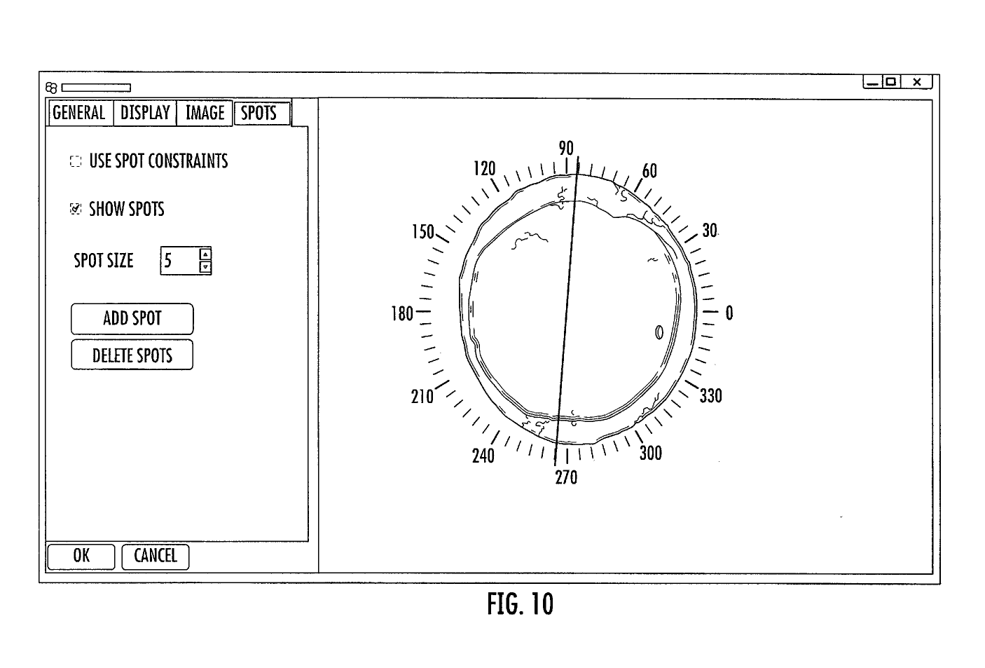

FIG. 10. Spots tab for retro-illumination editor.

It is to be understood that while a certain form of the invention is illustrated, it is not to be limited to the specific form or arrangement herein described and shown. It will be apparent to those skilled in the art that various changes may be made without departing from the scope of the inventions and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A method of measuring IOL orientation by use of retro-illumination and eye front surface feature registration comprising the steps of:
retro-illumination by use of a beacon from an ocular wavefront path to obtain an IOL image of an eye;
detecting registration features within the limbus contour and the outer most ring of said eye;
comparing point correspondences between two sets of registration features;
determining the rotational angle between said point references;
wherein retro-illumination imaging ensures that the principal axes of the IOL are positioned for eye image registration and determine a cyclorotation angle to correct the reporting of eye image registration at certain axes.

2. The method of measuring IOL orientation according to claim 1 wherein said retro-illumination of an eye to obtain a toric IOL image is further defined as acquiring a first image formed by a near IR beam of light that forms a diffuse reflection at the retina and the light is directed out of the eye filling the entrance pupil and back illuminating the toric IOL within the eye wherein light from the back lit toric IOL forms said first image on a camera sensor and a acquiring a second image where said corneal topography rings are illuminated, the light reflects off the front of the eye including the iris and sclera and forms said second image on a camera sensor.

3. The method of measuring IOL orientation according to claim 2 wherein said second image acquired is used to align the retro-illumination image data for another exam using features common to both images.

4. The method of measuring IOL orientation according to claim 1 wherein said retro-illumination image can include a toric IOL, phakic toric IOL, custom IOL, multifocal IOL, or other optical or mechanical features in the eye.

5. The method of measuring IOL orientation according to claim 1 wherein said retro-illumination image can be used to measure and document features such as cataract or other ocular changes at the IOL/crystalline plane.

6. The method of measuring IOL orientation according to claim 1 wherein said retro-illumination is through NIR or visible light.

7. The method of measuring IOL orientation according to claim 1 wherein said registration features are selected from the group of: corner features, blood vessels, forks or artificially placed marks.

8. The method of measuring IOL orientation according to claim 1 wherein said registration features have nearly a perpendicular image gradient.

9. The method of measuring IOL orientation according to claim 1 wherein said retro-illumination image is acquired from a digital camera attached to a surgical microscope.

10. The method of measuring IOL orientation according to claim 1 wherein said retro-illumination image is acquired from a video camera attached to a surgical microscope.

11. The method of measuring IOL orientation according to claim 10 wherein said retro-illumination image acquired by said video camera is a live image and used to identify toric IOL marks and for generating an orientation graphic display.

12. The method of measuring IOL orientation according to claim 1 including the step of capturing a live image through an operative microscope to identify toric IOL marks and generating an orientation graphic display

13. The method of measuring IOL orientation according to claim 1 wherein features in the iris pattern or on the sclera are identified to determine the rotation of the eye between image captures to align data between two eye exams taken apart to ensure axis measurements are to the lens and not to the eye's cyclorotation.

14. The method of measuring IOL orientation according to claim 1 including the step of storing said interactively selected points with an exam image and comparing said exam image to other images to be registered.
